(19) United States
(12) Patent Application Publication
(10) Pub. No.: US 2023/0191050 A1
(43) Pub. Date: Jun. 22, 2023

(54) FILTERED RESUSCITATION DEVICE

(71) Applicant: Adrian Salas, La Mesa, CA (US)

(72) Inventor: Adrian Salas, La Mesa, CA (US)

(21) Appl. No.: 17/996,192

(22) PCT Filed: Apr. 22, 2021
(86) PCT No.: PCT/US2021/028684

§ 371 Date: Oct. 13, 2022

Related U.S. Application Data
(60) Provisional application No. 63/016,103, filed on Apr. 27, 2020.

(51) Int. Cl. A61M 16/00; A61M 16/10; A61M 16/08

(52) U.S. Cl. CPC A61M 16/0084; A61M 16/0875; A61M 16/1065

(57) ABSTRACT

Filtered bag-valve mask resuscitators are disclosed. Proper ventilation can be delivered to patients with significantly reduced risk of exposing healthcare providers to severe airborne diseases, while not compromising the quality of care and allowing the option of using aerosolized medications. A filtered bag-valve mask resuscitator can also include an angled segment having a lumen configured to deliver gases to and/or from a filter.

### BACKGROUND

### Field

Some aspects of the invention relate to a filtered resuscitation device.

### Description of the Related Art

When a patient is being ventilated, such as by healthcare personnel including Emergency Medical Services (EMS) providers, physicians, nurses, and respiratory therapists for example, the air being exhaled is not filtered and possibly exposing others to any contagious airborne diseases the patient may have, including but not limited to respiratory pathogens including viruses (including but not limited to coronaviruses, e.g., COVID-19, influenza, parainfluenza, varicella, measles, mumps, enterovirus, rhinovirus, adenovirus, respiratory syncytial virus, norovirus, etc.); bacterial diseases (including tuberculosis, diphtheria, pertussis, and anthrax), fungal diseases (including but not limited to coccidiomycosis, blastomycosis, or histoplasmosis) and the like.

### SUMMARY

Devices that can advantageously provide proper ventilation to patients with significantly reduced risk of exposing healthcare providers to severe airborne pathogens/diseases, while not compromising the quality of care and allowing the option of using aerosolized medications are disclosed herein.

In some embodiments, disclosed herein is a filtered resuscitation device, comprising any number of: an inflatable bag comprising an enclosed volume; a manifold fluidly connected to a first end of the inflatable bag, the manifold comprising a gases inlet and a gases outlet; a filter fluidly connected to the manifold, the filter comprising an inlet or outlet comprising an angled bend segment comprising at least one lumen configured to deliver gases to and/or from the filter, an elongate flexible tubing connected to the filter; a multi-adapter connected to an end of the flexible tubing; and/or a patient interface configured to be attached to the multi-adapter and configured to deliver inspiratory gases to a patient and move expiratory gases away from the patient. In some embodiments, the filter is configured to reduce the number of pathogens in the expiratory gases such that the pathogens are trapped in the filter and do not flow to the outside environment. In some embodiments, the inflatable bag, manifold, filter, elongate flexible tubing, multi-adapter, and patient interface comprise a gases flow path.

In some embodiments, the angled bend has an angle of about 90 degrees with respect to the outlet of the manifold.

In some embodiments, the angled bend has an acute, right, or oblique angle with respect to the outlet of the manifold.

In some embodiments, the angled bend has an angle of between about 30 degrees and about 150 degrees with respect to the outlet of the manifold.

In some embodiments, the angled bend has an angle of between about 60 degrees and about 120 degrees with respect to the outlet of the manifold.

In some embodiments, a longitudinal axis of the inflatable bag is substantially parallel to a longitudinal axis of an outlet of the filter.

In some embodiments, a longitudinal axis of the inflatable bag is substantially parallel to a longitudinal axis of the elongate flexible tubing.

In some embodiments, the gases flow path is a monolumen.

In some embodiments, the gases flow path comprises a first inspiratory lumen configured to deliver inspired gases to the patient, and a second expiratory lumen configured to deliver expiratory gases away from the patient.

In some embodiments, the filter comprises a HEPA filter, a ULPA filter, and/or a carbon filter.

In some embodiments, the patient interface comprises an endotracheal tube, a tracheostomy tube, or supraglottic airway devices (e.g., a King airway, or a Combitube for example).

In some embodiments, the patient interface comprises a laryngeal mark airway mask.

In some embodiments, the patient interface comprises an oronasal face mask.

In some embodiments, the manifold comprises an expiratory valve.

In some embodiments, the manifold comprises a PEEP valve.

In some embodiments, at least some of the components of the filtered resuscitation device are integrally formed with each other.

In some embodiments, each of the components of the filtered resuscitation device are integrally formed with each other.

In some embodiments, disclosed herein is a filtered resuscitation device, comprising: a gases pump; a manifold fluidly connected to a first end of the gases pump, the manifold comprising a gases inlet and a gases outlet; a filter fluidly connected to the manifold, the filter comprising an inlet or outlet comprising an angled bend segment comprising at least one lumen configured to deliver gases to and/or from the filter; and a patient interface configured to deliver inspiratory gases to a patient and move expiratory gases away from the patient. The filter can be configured to reduce the number of pathogens in the expiratory gases such that the pathogens are trapped in the filter and do not flow to the outside environment. The gases pump, filter, and patient interface comprise a continuous gases flow path.

In some embodiments, the angled bend has an angle of about 90 degrees with respect to the outlet of the manifold.

In some embodiments, the angled bend has an acute, right, or oblique angle with respect to the outlet of the manifold.

In some embodiments, the angled bend has an angle of between about 30 degrees and about 150 degrees with respect to the outlet of the manifold.

In some embodiments, the angled bend has an angle of between about 60 degrees and about 120 degrees with respect to the outlet of the manifold.

In some embodiments, a longitudinal axis of the inflatable bag is substantially parallel to a longitudinal axis of an outlet of the filter.

In some embodiments, the gases pump comprises an inflatable BVM bag.

In some embodiments, the gases pump comprises a bellows.

In some embodiments, the gases pump comprises a ventilator.

In some embodiments, the gases pump comprises a CPAP mechanism.

In some embodiments, the gases pump comprises a BIPAP mechanism.

In some embodiments, a resuscitation device comprises any one or more of the embodiments described in the disclosure.

In some embodiments, a method for resuscitating a patient comprises any one or more of the embodiments described in the disclosure.

### BRIEF DESCRIPTION OF THE DRAWINGS

FIG. 1 illustrates an embodiment of a respiratory circuit of a resuscitation system.

FIG. 2 illustrates a close-up view of a filter with an angled bend, according to some embodiments.

FIG. 3 illustrates an embodiment of a filtered resuscitation device in a collapsed, potentially packaged state.

FIG. 4 illustrates side and perspective schematic views of an embodiment of a multi-adapter.

FIG. 5 schematically illustrates an embodiment of a conduit, e.g., an elongate tubing, such as nebulizer tubing.

FIG. 6 schematically illustrates an embodiment of a gases pump, such as a BVM bag.

### DETAILED DESCRIPTION

In some embodiments, disclosed herein is a filtered resuscitation device that can be used either with bag-valve mask (BVM) resuscitators, or automated mechanical ventilators in some cases. The exhaled air of a patient can be filtered using one or more filters, such as an in-line filter, minimizing exposure to medical personnel, while still in some cases allowing the availability of positive end-expiratory pressure (PEEP) devices when using a BVM resuscitator.

In some embodiments, an emergency ventilation and/or resuscitation system can include any number of the following features: a gas pump (e.g., a BVM bag or mechanical ventilation unit, CPAP unit, or BI/PAP unit for example), a filter comprising an angled segment, a conduit (e.g., nebulizer tubing), one or more adapters, and a patient interface, such as a sealing or non-sealing oral, nasal, or oral and nasal patient interface (e.g., a BVM mask), or an endotracheal tube or laryngeal mask airway in other embodiments. The patient interface could also be, for example, a tracheostomy tube, or supraglottic airway devices (e.g., a King airway, or a Combitube for example).

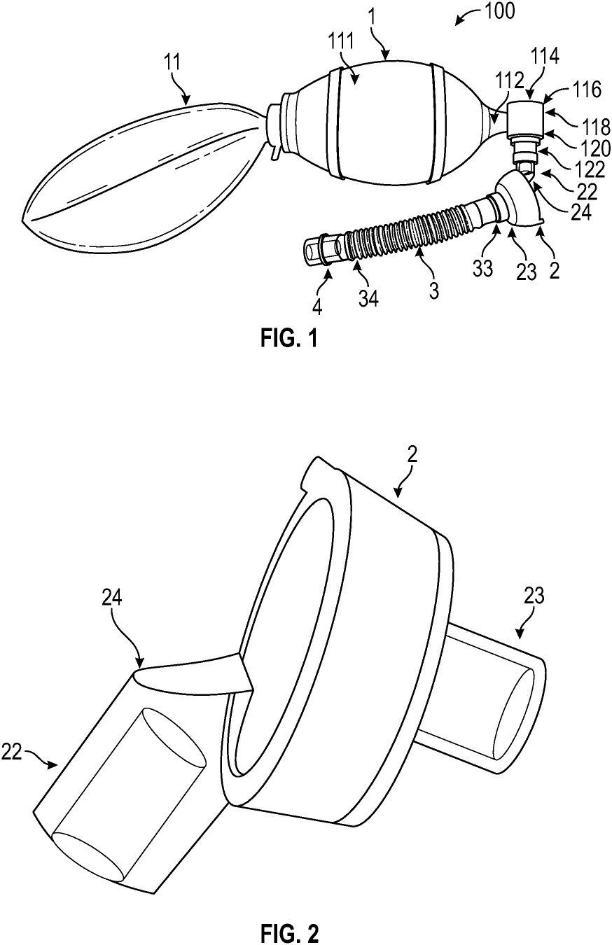

FIG. 1 illustrates an embodiment of a respiratory circuit of a resuscitation system 100. The gases pump 1, which can be a BVM bag in some embodiments, can provide delivery of gases to a patient, such as oxygen, room air, and the like. The gases pump 1 can include an inlet for connection to a gases source (illustrated schematically as 11), which can be simply atmospheric room air, or an enclosed source such as an oxygen tank, for example. The gases pump 1 can also include a movable wall 111 such as a bag, bellows, or other structure to allow for expansion and contraction of at least a portion of the gases pump 1 (e.g., BVM bag) to allow for gases movement to the patient’s airway. The gases pump 1 can also include an outlet 112 configured to a manifold 114 which is configured to attach to a filter 2. The manifold 114 can include, for example, any number of a PEEP valve 116, a pop-off valve 118, and an expiratory valve 120.

The filter 2 can, for example, include any number of a high efficiency particulate air (HEPA) filter, an ultra-flow particulate air (ULPA) filter, an activated carbon filter, a bacterial/viral filter, and the like. In some embodiments, the filter can generally be configured to capture particles of about or at least about 0.1, 0.2, 0.3, 0.4, 0.5, 0.6, 0.7, 0.8, 0.9, 1, 1.5, 2, or 3 microns or more or less, for example. The filter 2 can be positioned in-line between the manifold 114 and the inlet 33 of a conduit 3 (e.g., nebulizer tubing), and be configured to filter one or more (e.g., both) inspiratory and expiratory airflow. The filter 2 can also include an inspiratory port 22 directly connected to a first side of the filter 2 and an expiratory port 23 directly connected to a second side of the filter 2, e.g., for embodiments including a monolumen respiratory circuit, although multi-lumen (e.g., dual lumen embodiments that separate inspiratory and expiratory flow and still pass through the filter 2) embodiments are also contemplated in other embodiments. The inspiratory port 22 can be connected (e.g., integrally formed with or removably connected to) an outlet of the manifold 114. The expiratory port 23 can be connected (e.g., integrally formed with or removably connected to) to the conduit/elongate tubing 3. In some cases, integrally formed connections can advantageously save precious deployment time.

The filter 2, e.g., inspiratory port segment 22 can include an angled bend 24 in the tubing, as illustrated. The angled bend 24 can be a right angle (e.g., about 90 degrees). In some embodiments, the angled bend 24 can be an acute angle, a right angle, or an obtuse angle with respect to the outlet of the manifold 114. The angle can be, for example, greater than 0 degrees and less than 180 degrees, between about 45 degrees and about 135 degrees, between about 60 degrees and about 120 degrees, between about 75 degrees and about 105 degrees, about, at least about, or no more than about 10, 20, 30, 40, 50, 60, 70 80, 90, 100, 110, 120, 130, 140, 150, 160, or 170 degrees, or ranges including any two of the foregoing values. In some embodiments, the angled bend 24 can be on the expiratory port segment 23 of the filter 2 instead of, or in addition to the inspiratory port segment 22. In some embodiments, the angled bend 24 of the filter 2 can advantageously allow for a change of direction of the conduit reducing storage space required when packaged, and also protect the filter 2 and the assembly 1 from damage when in use and/or in the collapsed stored position, in an EMS bag for example.

The expiratory port 23 of the filter 2 can be directly connectable (e.g., integrally formed with or removably connected to) a first end 33 of the conduit 3, such as nebulizer tubing. The nebulizer tubing 3 can be a flexible tube with corrugations in some embodiments, and can axially lengthen and shorten similar to an accordion. The nebulizer tubing 3 can connect to a nebulizer port, such as a T-piece (not shown), e.g., at either end, to allow for the use of hand-held nebulizers in-line with the respiratory circuit 100. The T-piece can include a one-way valve to allow for the delivery of aerosolized medications into the circuit but prevent pathogens from exiting the circuit into the atmosphere. The nebulizer tubing 3 can have, in some cases, a fully stretched or unstretched length of, for example, between about 2 inches and about 6 inches, between about 2 inches and about 10 feet, between about 6 inches and about 10 feet, such as about 2 inches, about 6 inches, 1 foot, 2 feet, 3 feet, 4 feet, 5 feet, 6 feet, 7 feet, 8 feet, 9 feet, or 10 feet, or ranges including any two of the foregoing values, and a diameter of between about 2 cm and about 25 cm, or between about 5 cm and about 15 cm in some cases.

The second end 34 of the conduit 3 can be connected to a patient interface (not shown in FIG. 1) via an adapter 4, such as a 15 mm inner diameter (ID)/22 mm outer diameter (OD) multi-adapter for example. The patient interface can be, for example, a BVM face mask or other interfaces as disclosed elsewhere herein for example. In some cases, the nebulizer tubing 3 can be advantageous in allowing for distance, space, and maneuverability between itself and the BVM mask (not illustrated), and to reduce the risk that debris (respiratory debris such as mucus plugs for example, or GI debris such as aspiration/vomitus) will reach the filter. Another advantage is that such a setup allows the BVM manifold to remain free and available for other attachments/devices (e.g., the PEEP valve).

FIG. 2 illustrates a close-up view of a filter 2 with an angled bend 24, according to some embodiments. Filter 2 includes inspiratory port 22, angled bend 24, and expiratory port 23 that can be, for example as described elsewhere herein.

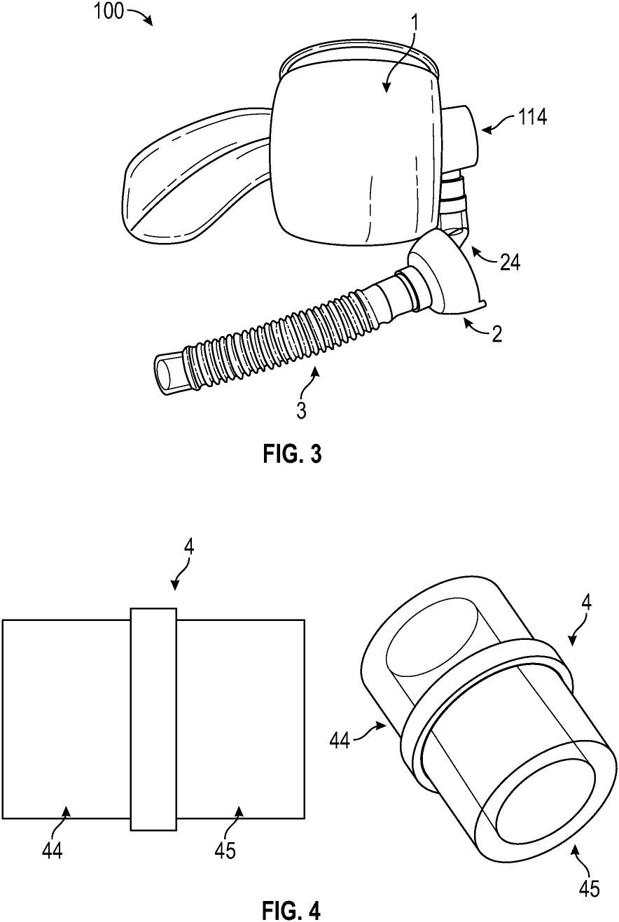

FIG. 3 illustrates an embodiment of a filtered resuscitation device 1 in a collapsed, potentially packaged state. As illustrated, in the packaged state as well as in the ready-for-use state of FIG. 1, the filter 2 and nebulizer tubing 3 of the respiratory circuit 100 can be parallel or substantially parallel to the longitudinal axis of BVM bag 1 and the flow path within the BVM bag 1. For example, the longitudinal axis of the tubing 3 may be positioned at an angle of 0 (e.g., parallel to), 5, 10, 15, 20, 25, 30, 35, 40, 45, or 50 degrees with respect to the longitudinal axis of the gasses pump 1. In some cases, the longitudinal axis of the tubing 3 may be positioned at an angle of less than, or less than or equal to 5, 10, 15, 20, 25, 30, 35, 40, 45, or 50 degrees with respect to the longitudinal axis of the gasses pump 1.

FIG. 4 illustrates side and perspective schematic views of a multi-adapter 4 that can be as previously described, with an outer diameter segment 45 and an inner diameter segment 44 less than that of the outer diameter segment 45. The outer diameter 45 can be, for example, about 22 mm while the inner diameter 44 can be, for example, about 15 mm in some cases, although other larger or smaller values are also possible.

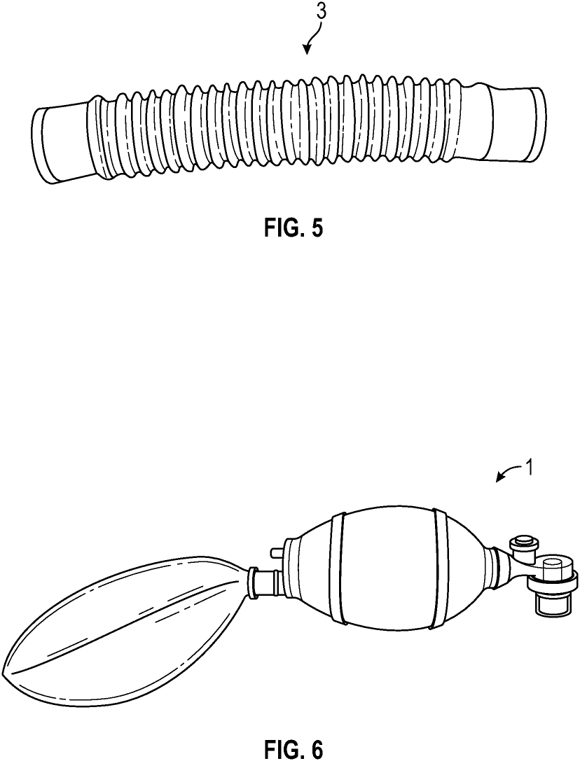

FIG. 5 schematically illustrates an embodiment of a conduit 3, e.g., an elongate tubing, such as nebulizer tubing that can be as described, for example, elsewhere herein.

FIG. 6 schematically illustrates an embodiment of a gases pump, such as a BVM bag 1, for example, that can be as described, for example, elsewhere herein.

Any one or more of the components of the resuscitation system 100 may be independently formed and attached to each other, or integrally formed with each other, such that they form a single unit. Independently formed components may be attached and detached from one another. Air-tight seals between coupled components may be achieved by providing one or more compression fittings, seals, gaskets, and/or o-rings at the junctions of coupled components of the resuscitation system 100. Integrally formed components may be formed as a single unit, for example, by forging, molding, welding, gluing, or otherwise bonding together one or more components of the resuscitation system 100.

For example, in some implementations, the multi-adapter 4 may be integrally formed with the tubing conduit 3, and/or the tubing conduit 3 may be integrally formed with the filter 2, and/or the filter 2 may be integrally formed with the gasses pump 1.

In some embodiments, a method of use of an embodiment of a resuscitation system will now be described. When the BVM is squeezed, gases, e.g., air / oxygen passes through the filter down the nebulizer tubing and into any airway device the multi-adapter is connected to. Once the patient begins to exhale, the expiratory gases from the patient then travels back up through the multi-adapter, the nebulizer tubing, and then contacts the filter which filters out any particulates that the filter is configured for, prior to the exhaled air reaching the BVM exhalation port and into the ambient atmosphere exposing those in the area to safe, filtered air from a patient’s exhalation.

In some embodiments, the filter can be moved such that it is directly adjacent to the patient interface and the multi-adapter. In some embodiments, the conduit (e.g., nebulizer tubing) and/or the multi-adapter is not required. In some embodiments, the distal end of the conduit (e.g., nebulizer tubing) can have the same or substantially the same ID and OD as the connector to the patient interface, rendering the multi-adapter unnecessary.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “delivering bag-mask ventilation” includes “instructing the delivery of bag-mask ventilation.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10% = 10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A filtered resuscitation device, comprising:
an inflatable bag comprising an enclosed volume;
a manifold fluidly connected to a first end of the inflatable bag, the manifold comprising a gases inlet and a gases outlet;
a filter fluidly connected to the manifold, the filter comprising an inlet or outlet comprising an angled bend segment comprising at least one lumen configured to deliver gases to and/or from the filter,
an elongate flexible tubing connected to the filter;
a multi-adapter connected to an end of the flexible tubing; and
a patient interface configured to be attached to the multi-adapter and configured to deliver inspiratory gases to a patient and move expiratory gases away from the patient,
wherein the filter is configured to reduce the number of pathogens in the expiratory gases such that the pathogens are trapped in the filter and do not flow to the outside environment,
wherein the inflatable bag, manifold, filter, elongate flexible tubing, multi-adapter, and patient interface comprise a gases flow path.

2. The device of claim 1, wherein the angled bend has an angle of about 90 degrees with respect to the outlet of the manifold.

3. The device of any of the preceding claims, wherein the angled bend has an acute, right, or oblique angle with respect to the outlet of the manifold.

4. The device of any of the preceding claims, wherein the angled bend has an angle of between about 30 degrees and about 150 degrees with respect to the outlet of the manifold.

5. The device of any of the preceding claims, wherein the angled bend has an angle of between about 60 degrees and about 120 degrees with respect to the outlet of the manifold.

6. The device of any of the preceding claims, wherein a longitudinal axis of the inflatable bag is substantially parallel to a longitudinal axis of an outlet of the filter.

7. The device of any of the preceding claims, wherein a longitudinal axis of the inflatable bag is substantially parallel to a longitudinal axis of the elongate flexible tubing.

8. The device of any of the preceding claims, wherein the gases flow path is a monolumen.

9. The device of any of the preceding claims, wherein the gases flow path comprises a first inspiratory lumen configured to deliver inspired gases to the patient, and a second expiratory lumen configured to deliver expiratory gases away from the patient.

10. The device of any of the preceding claims, wherein the filter comprises a HEPA filter.

11. The device of any of the preceding claims, wherein the filter comprises a ULPA filter.

12. The device of any of the preceding claims, wherein the filter comprises a carbon filter.

13. The device of any of the preceding claims, wherein the patient interface comprises an endotracheal tube.

14. The device of any of the preceding claims, wherein the patient interface comprises a laryngeal mark airway.

15. The device of any of the preceding claims, wherein the patient interface comprises an oronasal face mask.

16. The device of any of the preceding claims, wherein the manifold comprises an expiratory valve.

17. The device of any of the preceding claims, wherein the manifold comprises a PEEP valve.

18. The device of any of the preceding claims, wherein at least some of the components of the filtered resuscitation device are integrally formed with each other.

19. The device of any of the preceding claims, wherein each of the components of the filtered resuscitation device are integrally formed with each other.

20. A filtered resuscitation device, comprising:
a gases pump;
a manifold fluidly connected to a first end of the gases pump, the manifold comprising a gases inlet and a gases outlet;
a filter fluidly connected to the manifold, the filter comprising an inlet or outlet comprising an angled bend segment comprising at least one lumen configured to deliver gases to and/or from the filter; and
a patient interface configured to deliver inspiratory gases to a patient and move expiratory gases away from the patient,
wherein the filter is configured to reduce the number of pathogens in the expiratory gases such that the pathogens are trapped in the filter and do not flow to the outside environment,
wherein the gases pump, filter, and patient interface comprise a continuous gases flow path.

21. The device of claim 20, wherein the angled bend has an angle of about 90 degrees with respect to the outlet of the manifold.

22. The device of claim 20 or 21, wherein the angled bend has an acute, right, or oblique angle with respect to the outlet of the manifold.

23. The device of any of claims 20-22, wherein the angled bend has an angle of between about 30 degrees and about 150 degrees with respect to the outlet of the manifold.

24. The device of any of claims 20-23, wherein the angled bend has an angle of between about 60 degrees and about 120 degrees with respect to the outlet of the manifold.

25. The device of any of claims 20-24, wherein a longitudinal axis of the inflatable bag is substantially parallel to a longitudinal axis of an outlet of the filter.

26. The device of any of claims 20-25, wherein the gases pump comprises an inflatable BVM bag.

27. The device of any of claims 20-26, wherein the gases pump comprises a bellows.

28. The device of any of claims 20-27, wherein the gases pump comprises a ventilator.

29. The device of any of claims 20-28, wherein the gases pump comprises a CPAP mechanism.

30. The device of any of claims 20-29, wherein the gases pump comprises a BIPAP mechanism.

31. A resuscitation device, comprising any one or more of the embodiments described in the disclosure.

32. A method for resuscitating a patient, comprising any one or more of the embodiments described in the disclosure.
